(19) United States
(12) Patent Application Publication
(10) Pub. No.: US 2026/0324997 A1
(43) Pub. Date: Oct. 22, 2026

(54) SYSTEMS AND METHODS FOR NON-INVASIVE DETERMINATION OF ABSOLUTE BLOOD LOSS AND HEMODYNAMIC PARAMETERS WITH OPTICAL BLOOD FLOW SENSOR

(71) Applicant: Washington University, St. Louis, MO (US)

(72) Inventors: Francesca Bonetta-Misteli, St. Louis, MO (US); Peinan Zhao, St. Louis, MO (US); Christine O'Brien, St. Louis, MO (US); Antonina Frolova, St. Louis, MO (US); David Monks, St. Louis, MO (US); Leonid Shmuylovich, St. Louis, MO (US)

(21) Appl. No.: 19/458,073

(22) Filed: Jan. 23, 2026

Related U.S. Application Data
(60) Provisional application No. 63/749,078, filed on Jan. 24, 2025.

(51) Int. Cl. A61B 5/026; A61B 5/00; A61B 5/02; A61B 5/024; G16H 10/60; G16H 50/30

(52) U.S. Cl. CPC A61B 5/0261; A61B 5/02028; A61B 5/02416; A61B 5/02438; A61B 5/681; A61B 5/7275; A61B 5/746; G16H 10/60; G16H 50/30

(57) ABSTRACT

A system comprising a coherent light-based sensor operable to generate a signal representing perfusion of an individual over a first time period and processing circuitry configured to determine a hemodynamic state of the individual over the first time period based on a set of features extracted from the signal.

### CROSS-REFERENCE TO RELATED APPLICATIONS

This application claims the benefit of U.S. Provisional Patent Application No. 63/749,078, filed Jan. 24, 2025, which is incorporated herein by reference in its entirety.

### STATEMENT REGARDING FEDERALLY SPONSORED RESEARCH & DEVELOPMENT

This invention was made with government support under HD103954 and HL179205 awarded by the National Institutes of Health and DGE2139839 awarded by the National Science Foundation. The government has certain rights in the invention.

### TECHNICAL FIELD

The present disclosure relates to a device for determining a hemodynamic state of an individual. Particularly, but not exclusively, the present disclosure relates to a device using a coherent light-based sensor to generate signals for determining a hemodynamic state of an individual; more particularly, but not exclusively, features are extracted from a coherent light-based signal to determine hemodynamic state.

### BACKGROUND

The accurate and timely monitoring of an individual's hemodynamic state is critical across numerous clinical settings. This is particularly important in high-stakes environments such as obstetrics, surgery, critical care, trauma, and during anesthesia. In these settings, undetected hemodynamic instability, often resulting from hemorrhage, is a leading cause of preventable morbidity and mortality.

For example, in obstetrics, postpartum hemorrhage (PPH), defined as the loss of 1 L of blood or more within 24 hours after birth, is often identified through visual estimation of blood loss; a technique which is shown to underestimate the amount of blood lost. This is particularly problematic since PPH is the leading cause of maternal mortality worldwide with an estimated 14 million cases each year resulting in 130,000 deaths. PPH is, however, the most preventable cause of maternal mortality provided onset of PPH can be identified early. This is especially important in low-resource settings that often have low blood stores for transfusion, relying primarily on early pharmacologic treatment. Similar challenges exist in other medical domains. In surgical and trauma settings, internal or external bleeding can be difficult to quantify accurately in real-time. Clinicians often rely on lagging indicators such as changes in blood pressure or heart rate, which only manifest after the body's compensatory mechanisms begin to fail. In critical care and under anesthesia, patients are often hemodynamically fragile, and subtle changes in cardiovascular function need to be detected early to prevent adverse outcomes. Conventional monitoring can be intermittent and may not capture the rapid onset of instability.

There is thus a need for improved approaches for the early and accurate determination of hemodynamic state.

### SUMMARY OF DISCLOSURE

According to an aspect of the present disclosure there is provided a system including: a coherent light-based sensor operable to generate a signal representing perfusion of an individual over a first time period; and processing circuitry configured to determine a hemodynamic state of the individual over the first time period based on a set of features extracted from the signal.

According to a further aspect of the present disclosure there is provided a method including: obtaining, by processing circuitry, a signal generated by a coherent light-based sensor, wherein the signal represents perfusion of an individual over a first time period; and determining, by processing circuitry, a hemodynamic state of the individual over the first time period based on a set of features extracted from the signal.

According to an additional aspect of the present disclosure there is provided a non-transitory computer readable medium including instructions which, when executed by processing circuitry, cause the processing circuitry to perform the method comprising: obtaining, by processing circuitry, a signal generated by a coherent light-based sensor, wherein the signal represents perfusion of an individual over a first time period; and determining, by processing circuitry, a hemodynamic state of the individual over the first time period based on a set of features extracted from the signal.

Advantages will become more apparent to those of ordinary skill in the art from the following description of the preferred examples which have been shown and described by way of illustration. As will be realized, the present examples may be capable of other and different examples, and their details are capable of modification in various respects. Accordingly, the drawings and description are to be regarded as illustrative in nature and not as restrictive.

### BRIEF DESCRIPTION OF THE DRAWINGS

Examples of the present disclosure will now be described, by way of example only, and with reference to the accompanying drawings, in which:

FIG. 1 shows a device according to an aspect of the present disclosure.

FIG. 2 illustrates morphological feature extraction from a coherent light-based waveform according to embodiments of the present disclosure.

FIG. 3A illustrates the generation of a baseline signal or waveform from a set of reference signals.

FIG. 3B illustrates the generation of relative features according to embodiments of the present disclosure.

FIG. 4A illustrates the true blood loss verse predicted blood loss using absolute features.

FIG. 4B illustrates the true blood loss verse predicted blood loss using relative features.

FIG. 5A illustrates the performance of estimating hemodynamic state of an individual using mean signal values obtained from a coherent light-based sensor.

FIG. 5B illustrates the performance of estimating hemodynamic state of an individual using features extracted from signals generated by a coherent light-based sensor.

FIG. 6A illustrates a method according to an aspect of the present disclosure.

FIG. 6B illustrates a method according to an embodiment of the present disclosure.

FIG. 7 illustrates a machine learning infrastructure.

FIG. 8 illustrates an example computing device.

### DETAILED DESCRIPTION

Existing methods for monitoring the hemodynamic state of an individual suffer from significant limitations that can lead to delayed diagnosis and treatment, particularly in cases of hemorrhage. Clinicians often rely on lagging indicators such as changes in heart rate or blood pressure, which may not manifest until after the body's compensatory physiological mechanisms, such as peripheral vasoconstriction, are overwhelmed. Furthermore, common methods for quantifying blood loss, such as visual estimation, are notoriously imprecise and prone to underestimation, especially in cases of internal bleeding. While non-invasive optical sensors based on photoplethysmography (PPG) exist, they are often unreliable in the low-perfusion states common during hemorrhage and are highly susceptible to inaccuracies caused by variations in skin pigmentation, which acts as a significant optical confounder. Conversely, gold-standard invasive techniques for measuring key parameters like cardiac output are too costly, complex, and carry inherent risks making them unsuitable for routine or widespread continuous monitoring.

The present disclosure helps address these problems by providing a device for determining the hemodynamic state of an individual based on features extracted from a signal generated by a coherent light-based sensor. The coherent light-based signal provides a much higher signal-to-noise ratio (SNR), works effectively in low-perfusion states, and is significantly less prone to inaccuracies caused by skin pigmentation. Moreover, the features extracted from such a signal captures subtle physiological changes that are direct indicators of the underlying cardiovascular state of the individual.

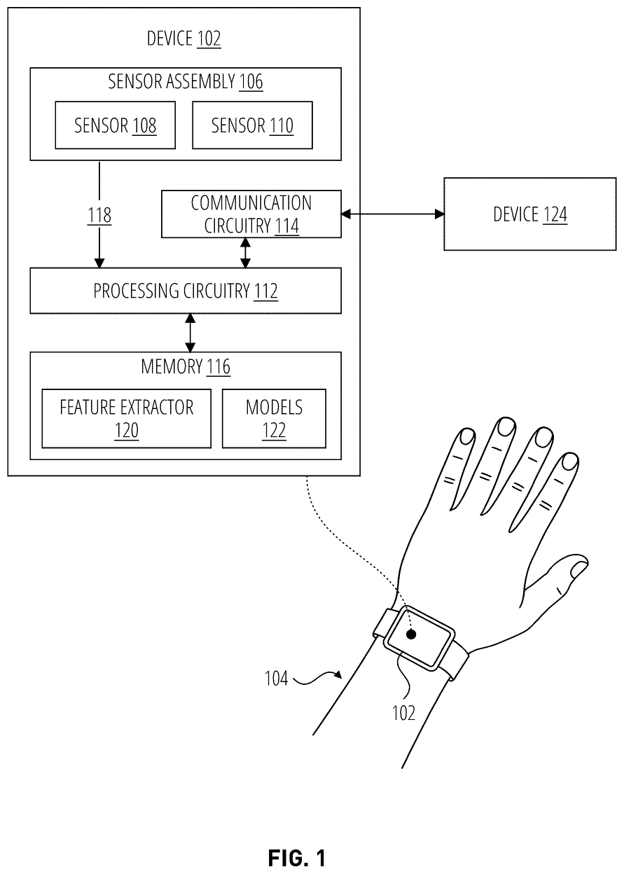

FIG. 1 shows a system according to an aspect of the present disclosure. The system includes a device 102, sometimes referred to as a first device.

The device 102 monitors the hemodynamic state of an individual 104 (e.g., a patient, subject, user, or the like). The device 102 comprises a sensor assembly 106 which includes a coherent light-based sensor 108 and, in some embodiments, also includes a hemoglobin sensor 110. The device 102 further comprises processing circuitry 112, communication circuitry 114, and memory 116. In some embodiments, the device 102 includes a housing that encloses the sensor assembly 106, the processing circuitry 112, the communication circuitry 114, and the memory 116. The housing may be any suitable housing, enclosure, case, covering or the like that encloses, protects, contains, holds, and/or supports the components of the device 102. The sensor assembly 106 is shown outputting a signal 118 which is generated from the coherent light-based sensor 108. The memory 116 comprises a feature extractor 120 and one or more models 122. FIG. 1 further shows a remote device 124, sometimes referred to as a second device, which is communicatively coupled to the device 102. The device 102 shown in FIG. 1 is a wearable device. However, the present disclosure is not limited to wearable devices only and other types of devices are envisaged (e.g., a clinical monitoring device or a sub-device of a physiological monitoring device).

In general, the device 102 is worn by the individual 104 to monitor the hemodynamic state of the individual 104 over a period of time. The coherent light-based sensor 108 generates the signal 118 which represents perfusion of the individual 104 over a first time period (e.g., 5 s, 10 s, 30 s, etc.). The processing circuitry 112 determines a hemodynamic state of the individual 104 over the first time period based on a set of features extracted from the signal 118 (e.g., using the feature extractor 120).

The device 102 may be a wearable device such as a wrist-worn wearable device or the like. The device 102 may alternatively be integrated into an existing device or wearable device such as a smart watch or wearable physiological monitor. In some embodiments, the housing or part of the housing is configured for facilitating wearing of the device 102 by the individual. That is, the housing may include, be attached to, or be configured for attachment to an elastic band, a strap, a watch-style wrist band, or the like. In some embodiments, the device 102 may be attached to the individual 104 rather than being worn as a wearable device, and the housing may be configured for such attachment. For example, the housing may include a skin appropriate adhesive layer for adhering the device 102 to the skin of the individual, may include a smooth surface for receiving a skin appropriate adhesive, may include one or more surfaces shaped and/or sized to conform to an individual's body, may be sized and/or shaped for insertion in a pocket in an adhesive or wrapped bandage, or the like. In some embodiments, the device 102 is attached to the individual by being placed against the skin of the individual and held in place by an elastic bandage, tape, a compression bandage, or any other suitable component for holding the device 102 against the skin of the individual. Although illustrated and discussed with respect to placement on the wrist of the individual, it should be understood that the device 102 may be positioned at any other location on the body of an individual where the device 102 may function as described herein. The skilled person will also appreciate that the device 102 shown in FIG. 1 is a simplification for brevity and ease of understanding. The device 102 may therefore include further features and/or components not shown in FIG. 1.

The sensor assembly 106 comprises a coherent light-based sensor 108 and optionally comprises a hemoglobin sensor 110. The coherent light-based sensor 108 is a perfusion sensor that illuminates tissue with coherent optical radiation and generates an output signal indicative of blood flow based on light received from the tissue. In various embodiments, the returned light is analyzed for speckle contrast and/or phase-or frequency-shift (Doppler) features to compute a signal indicative of blood-flow magnitude and/or dynamics. The coherent light-based sensor 108 may be one of: a laser speckle flow index (LSFI) sensor; a speckleplethysmography (SPG) sensor; a speckle contrast optical spectroscopy (SCOS) sensor; a dynamic light scattering (DLS) sensor; a speckle visible spectroscopy sensor. As will be described in more detail below, the signal 118 generated by the coherent light-based sensor 108 may be represented as a waveform of pulses (or heartbeats) from which features are extracted to determine the hemodynamic state of the individual 104. The hemoglobin sensor 110 utilizes short-wave infrared (SWIR) light to measure hemoglobin concentration of the individual 104. The hemoglobin sensor 110 may employ a 900 nm wavelength, which is strongly absorbed by hemoglobin, and a 1300 nm wavelength, which is strongly absorbed by water, to create a ratiometric measurement. The hemoglobin levels may be incorporated as a further feature provided to the models 122 to predict hemodynamic state of the individual 104. Advantageously, the use of the coherent light-based sensor 108 and/or the hemoglobin sensor 110 move away from the visible light spectrum-where melanin of the individual 104 is a significant confounding variable-and into the near-infrared (NIR) and short-wave infrared (SWIR) regions. In these regions, melanin's absorption is significantly lower, allowing the sensors to capture a high-quality signal directly from the underlying blood flow and composition thereby enabling the device 102 to be effectively used across a large population of individuals with differing skin tones. Additional details of sensors, sensor assemblies, and techniques, at least some of which may be used in embodiments of this disclosure may be found in U.S. Patent Application Publication No. 2024/0156355, U.S. Patent Application Publication No. 2025/0152021, and U.S. Patent Application Publication No. 2025/0064333, each of which is incorporated herein by reference in its entirety.

The processing circuitry 112 is configured to determine the hemodynamic state of the individual over the first time period based on a set of features extracted from the signal. Here, hemodynamic state may be understood as a representation of the cardiovascular condition of the individual 104 at a given time or over a predefined time period. The hemodynamic state comprises at least one value indicative of vascular and cardiac dynamics of the individual. The at least one value comprises one or more of: a value quantifying an amount of blood loss by the individual, a value quantifying a blood volume of the individual, a value indicative of onset of one or more of: preeclampsia; hemorrhage; postpartum hemorrhage, a value indicative of a degree of adequacy of tissue perfusion. Additionally, or alternatively, the hemodynamic state comprises at least one value indicative of a cardiac function of the individual over the first time period and/or at least one value indicative of a cardiovascular stability of the individual over the first time period. As will be described in more detail below, the one or more models 122 may be used to predict at least one of the above described values to encode the hemodynamic state of the individual 104.

In one embodiment, the processing circuitry 112 pre-processes the signal 118 prior to extracting the set of features. For example, the signal 118 may be bandpass filtered with a first-order finite impulse response filter using a passband of 0.5-10 Hz. The signal 118 comprises a waveform including one or more cardiac pulses (e.g., pulses, cardiac cycles, heartbeats). In general, a cardiac pulse may be understood as a signal or waveform which consists of a systolic phase identified by a systolic peak and a subsequent diastolic phase identified by a diastolic peak. The processing circuitry 112 may segment the signal 118 to extract or identify one or more cardiac pulses within the signal 118. The skilled person will appreciate that any suitable pulsatile segmentation algorithm may be used. In one implementation, a Fourier transform is used to determine the fundamental frequency of the signal 118 (which corresponds approximately to heart rate) and peak detection guided by the calculated frequency is used to extract the one or more cardiac pulses. The features used to determine the hemodynamic state may then be based on at least one cardiac pulse identified within the signal 118.

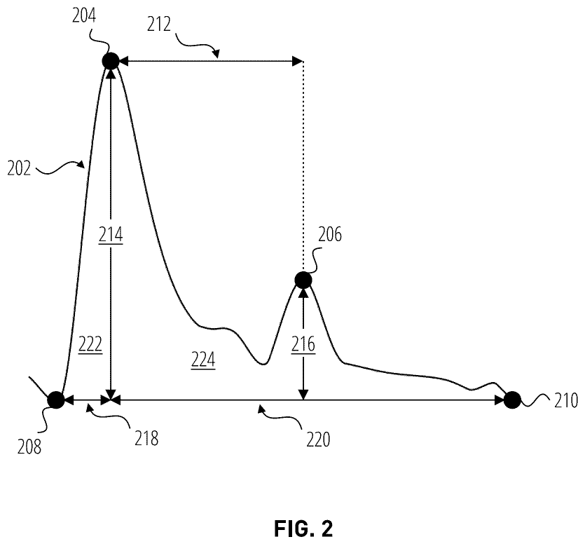

The set of features are extracted from the signal 118 using the feature extractor 120. As will be described in more detail in relation to FIG. 2 below, the feature extractor 120 extracts one or more features, such as morphological or pulsatile features, from one or more cardiac pulses present in the signal 118 based on keypoints identified from the one or more cardiac pulses. The features encode characteristics of the signal 118 over the first time period which in turn provide a compact and discriminative set of features which can be used to quantify the hemodynamic state of the individual over the first time period. Example features extracted from the signal 118 include a time delay, a systolic amplitude, a diastolic amplitude, an augmentation index, a total time, a systolic time, a diastolic time, a total area, a systolic area, a diastolic area, and a heart rate. The set of features extracted from the signal 118 thus describe or encode the shape, structure, and timing characteristics of the signal 118 and may be alternatively referred to as waveform features, morphological features, pulsatile features, or hemodynamic features. Additionally, or alternatively, the set of features may include frequency-based features determined from the signal 118. That is, the signal 118 may be transformed into the frequency domain via an algorithm such as fast Fourier transform (FFT), wavelet decomposition, short-time Fourier transform (STFT) or the like, and features extracted (e.g., the FFT coefficients, wavelet energy, dominant peak frequency, power spectral density (PSD), spectral bandwidth, etc.) to form the set of features. Additionally, or alternatively, the set of feats may include model-based features determined from the signal 118 by fitting the signal 118 to a model and the parameters of the model form the set of features. Additionally, or alternatively, the set of features may include statistical features of the signal 118, features associated with variability of the signal 118, and/or noise-based features of the signal 118 (e.g., features related to quality metrics of the signal 118).

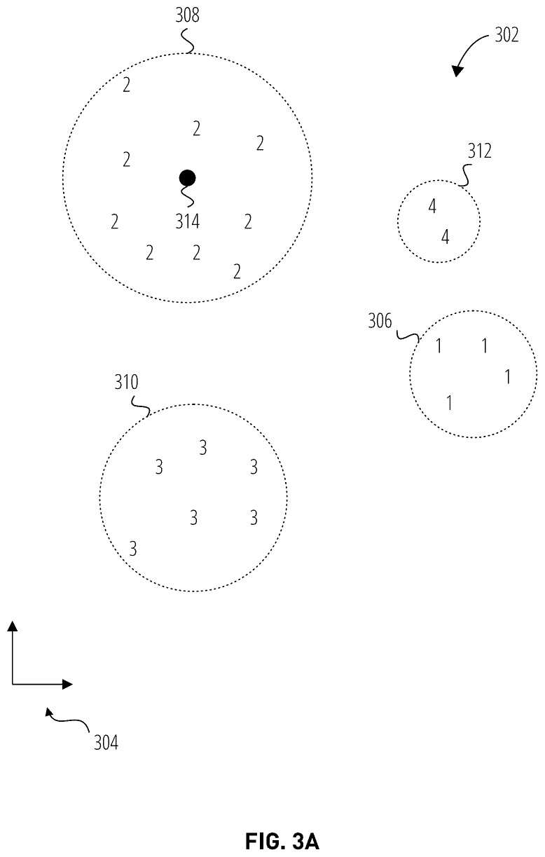

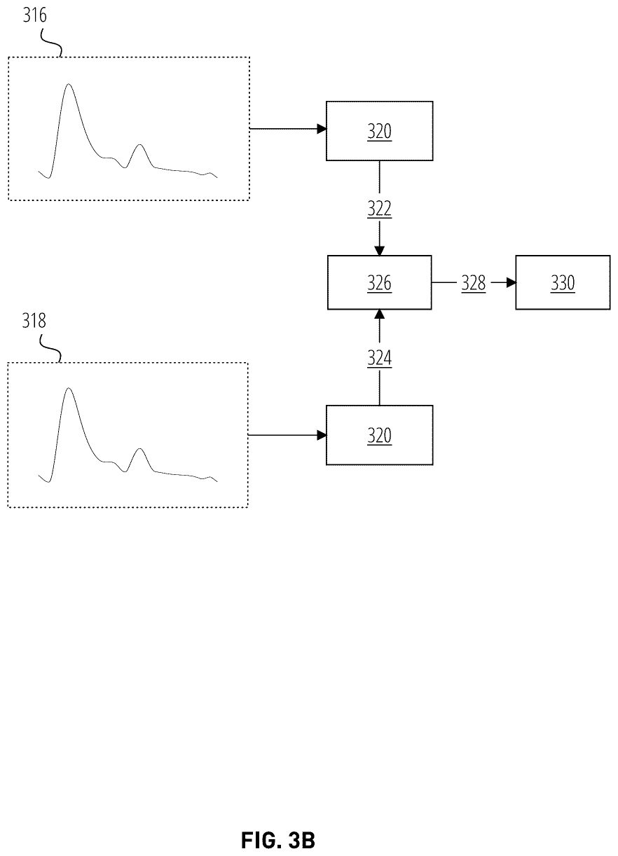

In one embodiment, the set of features are determined relative to a baseline signal for the individual 104. The baseline signal corresponds to a previously generated or obtained signal which encodes a characteristic hemodynamic state of the individual 104 (as described in more detail in relation to FIG. 3B below). For example, the baseline signal may be determined or obtained from the individual 104 prior to onset of any hemodynamic instability (e.g., whilst the individual 104 is in a steady state). The baseline signal may therefore be understood as an idealized signal which can be used to determine a change in hemodynamic response/state of the individual 104. The baseline signal may consist of a single pulse or waveform from which a reference set of features are extracted. These reference features may be stored in the memory 116 of the device 102. The processing circuitry 112 obtains the reference set of features (e.g., from the memory 116) and determines the output set of features from the reference set of features and the features extracted from the signal 118 (e.g., by calculating the percentage change between the two sets of features).

The processing circuitry 112 may be configured to perform a quality control (QC) process to filter the set of features. The quality control process comprises one or more QC tasks which are applied to each waveform segmented from the signal 118 to determine whether the features extracted from each waveform are suitable for use in determining hemodynamic state. The one or more QC tasks include a first task which removes features from the set of features which are associated with a waveform (i.e., a single cardiac cycle or heartbeat) having (1) a systolic peak occurring at a point in time after the identified diastolic peak of the waveform; and/or (2) a systolic peak having a lower amplitude than the identified diastolic peak. A second QC task removes features from the set of features which are associated with a waveform having a length (i.e., time) which does not fall within a range of expected length values. The range of expected length values are determined from the heartrate extracted from the signal 118 (e.g., the features are rejected if the length does not fall within 0.1 s of the expected length). A third QC task removes features from the set of features which are associated with a waveform which does not match, or substantially match, the shape of a reference waveform. The reference waveform may be a personalized or baseline waveform (as described in more detail below) or a synthetic signal or waveform indicating an ideal or reference pulse shape. In one implementation, the two waveforms are compared by normalizing amplitude to the range [0, 1] and normalizing the time axis using dynamic time warping. A correlation coefficient between the waveform under evaluation and the reference waveform is calculated and the associated features are excluded if the correlation coefficient is less than a predetermined threshold value (e.g., <0.9, <0.95, <0.97, etc.). In embodiments where accelerometer data is available (e.g., from an accelerometer sensor associated with, or incorporated into, the device 102), a fourth QC task removes features from the set of features which are associated with a waveform generated at a time point associated with a spike in the accelerometer data. Such a spike is typically indicative of motion which may introduce motion artefacts into the waveform data. Removing features associated with such waveforms therefore helps reduce the influence of motion artefacts.

To determine the hemodynamic state of the individual 104 over the first time period, the processing circuitry 112 is configured to process the set of features using one or more of the models 122. As the hemodynamic state of the individual 104 may be quantified or expressed using different values (e.g., a value indicative of vascular and cardiac dynamics of the individual, a value quantifying an amount of blood loss by the individual, etc.), the one or more models 122 typically comprise multiple statistical, machine learning, and/or deep learning models trained to predict values indicative of the hemodynamic state of the individual from morphological or pulsatile features. For example, in one example implementation, the one or more models 122 include a single statistical model which is trained to estimate from a set of features a value quantifying an amount of blood loss by the individual. In other implementations, multiple models may be used concurrently to estimate different values indicative of the hemodynamic state of the individual (e.g., a scalar value of blood volume, a categorical value of cardiovascular stability, etc.). The one or more models 122 may be trained on absolute feature values or relative feature values. Example prediction models suitable for use as the one or more models 122 include linear regression models, random forest models, and deep learning models.

In one embodiment, at least one of the one or more models 122 determine the hemodynamic state of the individual 104 based on the set of features and one or more demographic features and/or electronic health record (EHR) features related to the individual 104. Example demographic features include age, sex, ethnicity, and the like. Example EHR features include height, weight, previous diagnoses, indicators of cardiac devices, lab results, and the like. The demographic and/or EHR features related to the individual 104 may be obtained from an electronic medical record of the individual 104. Additionally, or alternatively, at least one or the one or more models 122 determine the hemodynamic state of the individual 104 based on Hb measurements obtained from the hemoglobin sensor 110.

In some situations, the set of features may comprise features extracted from multiple cardiac pulses present within the signal 118. In a simple example, if the features total time and augmentation index are determined from each pulse, then the set of features may comprise a total time value and an augmentation index value for multiple cardiac pulses within the signal 118. In this instance, the hemodynamic state may be determined as an aggregate or average prediction of hemodynamic state from the total time values and augmentation index values extracted from the multiple pulses.

The one or more models 122 are centrally trained on a large dataset of example sets of features with associated hemodynamic state values. In this way a single set of models may be deployed and/or used across multiple devices. In embodiments where relative features are used (i.e., the features provided to the model are relative to a baseline set of features), the use of relative features allows the personal hemodynamic response of an individual to be incorporated into the prediction without requiring a personalized prediction model to be trained for them.

In one embodiment, the processing circuitry 112 causes execution of an action based on the hemodynamic state of the individual 104. As such, the device 102 may continuously monitor the hemodynamic state of the individual and cause execution of an action when the hemodynamic state satisfies a criterion. For example, the criterion may be satisfied when the hemodynamic state is indicative of a hemorrhage, onset of a potential hemorrhage, or impending onset of a potential hemorrhage. As such, the present disclosure is able to predict the start of hemorrhage before it happens by detecting blood loss levels that are lower than an absolute threshold associated with the volume of blood loss associated with a hemorrhage. The criterion may be predetermined or may be dynamically adjusted based on criterion is based on one or more of: a baseline physiology of the individual; a rate of change in hemodynamic state of the individual; a compensation trajectory of the individual.

The action may cause the generation and presentation of an alert such as a visual alert, an audible alert, or a haptic alert. The alert can be executed on the device 102 and/or another device (e.g., the remote device 124, a second wearable device worn by a second individual such as a healthcare professional, a mobile device or tablet, a monitoring station, etc.). For example, when the hemodynamic state is indicative of a potential onset of a postpartum hemorrhage (PPH), a visual and audible alert may be executed/presented on a monitoring device used by a healthcare professional such as a nurse, midwife, or doctor. In this way, the healthcare professional is alerted to the onset of PPH at an early stage thereby enabling them to provide relevant treatment to improve the outcome of the individual.

Additionally, or alternatively, the action may update one or more parameters of the coherent light-based sensor 108. For example, if the hemodynamic state indicates potential hemodynamic instability (e.g., onset of a hemorrhage), then the sampling rate of the coherent light-based sensor 108 may be increased to increase the fidelity and resolution of the signal generated by the coherent light-based sensor 108 during a period of suspected instability. This helps efficiently manage power to the device 102 by ensuring that higher resolution signals which consume more power are reserved for periods of time in which the increased resolution may help identify hemodynamic instabilities and thus improve patient outcomes.

In one embodiment, the set of features and the hemodynamic state are determined by the remote device 124 (i.e., instead of by the device 102). In such an embodiment, the device 102 may be a low power device which generates the signal 118 and transmits the signal 118 to the device 102 for processing and action execution.

In some embodiments, the device 102 and/or the remote device 124 monitors changes in hemodynamic state of the individual 104 over multiple time periods. For example, the above described process of determining a hemodynamic state of the individual 104 may be repeated over a second time period (after the first time period) and a change in state determined. This can inform various clinical tasks and can indicate potential risk to the individual 104 by indicating the extent to which the individual 104 is compensating.

FIG. 2 illustrates feature extraction from a coherent light-based waveform according to embodiments of the present disclosure.

FIG. 2 shows a waveform 202, a first key point 204, a second key point 206, a third key point 208, and a fourth key point 210. FIG. 1 further shows a set of features 212-220 extracted from the waveform 202.

The waveform 202 corresponds to a coherent light-based waveform (e.g., a waveform generated by a coherent light-based sensor such as the coherent light-based sensor 108 shown in FIG. 1). The waveform 202 captures a single pulse, or cardiac cycle, of an individual and is used to determine features which encode the hemodynamic activity or state of the individual. The features described below may thus be considered hemodynamic features, morphological features, and/or pulsatile features.

The first key point 204 corresponds to the systolic peak of the waveform 202, the second key point 206 corresponds to the diastolic peak of the waveform 202, and the third key point 208 and the fourth key point 210 correspond to the systolic bases of the waveform 202. These keypoints provide reference points within the waveform 202 from which feature values which encode the shape, structure, and temporal properties of the waveform 202 can be extracted.

In one embodiment, the key points are detected using signal processing. The first key point 204 (i.e., the systolic peak) may be identified by determining the maximum value of the waveform 202. The second key point 206 (i.e., the diastolic peak) may be identified by calculating the first and second derivatives of the waveform 202 and identifying the point at which the second derivative reaches a minimum and the first derivative is zero. The third key point 208 and the fourth key point 210 (i.e., the first and second systolic base) may be identified by determining the peaks of the second derivative of the waveform 202 and confirming that these points are local minima using the first derivative of the waveform 202.

In another embodiment, the key points are detected using a trained deep learning model. A multi-layered U-net model is trained on a set of waveforms with manually selected systolic and diastolic points with Gaussian probability distributions to estimate probability distributions for both systolic and diastolic peaks within a waveform. The model will then output two probability curves for the systolic and diastolic peaks of the waveform.

The set of features may include a time delay feature 212, Δt_{del}. The time delay feature 212 may be calculated as the absolute difference between the first key point 204 and the second key point 206.

The set of features may include a systolic amplitude feature 214, x_{sys}. The systolic amplitude feature 214 may be calculated as the amplitude of the first key point 204 relative to a baseline value of the waveform 202. The baseline value may be the amplitude of the third key point 208, the amplitude of the fourth key point 210, or an average of these two amplitudes.

The set of features may include a diastolic amplitude feature 216, x_{dias}. The diastolic amplitude feature 216 may be calculated as the amplitude of the second key point 206 related to the baseline value of the waveform 202 (as described above).

The set of features may include an augmentation index value, Alx. The augmentation index value may be calculated as the ratio of the diastolic amplitude x_{dias}to the systolic amplitude x_{sys}; i.e., Alx=x_{dias}/x_{sys}. The set of features may include a total time value, Δtₜₒₜ. The total time value, Δtₜₒₜ may be calculated as the absolute difference in time between the third key point 208 and the fourth key point 210.

The set of features may include a systolic time value 218, Δt_{sys}. The systolic time value 218 Δt_{sys} may be calculated as the absolute difference in time between the first key point 204 and the third key point 208.

The set of features may include diastolic time value 220, Δt_{dias}. The diastolic time value 220 Δt_{dias} may be calculated as the absolute difference in time between the first key point 204 and the fourth key point 210.

The set of features may include a systolic area feature 222, A_{sys}. The systolic area feature 222 A_{sys} may be calculated as the area under the curve of the waveform 202 from the time point associated with the third key point 208 and the time point associated with the first key point 204.

The set of features may include a diastolic area feature 224, A_{dias}. The diastolic area feature 224 A_{dias}may be calculated as the area under the curve of the waveform 202 from the time point associated with the first key point 204 to the time point associated with the fourth key point 210.

The set of features may include a total area feature, Aₜₒₜ. The total area feature Aₜₒₜ may be calculated as the area under the curve of the waveform 202 between the third key point 208 and the fourth key point 210 (i.e., Aₜₒₜ=A_{sys}+A_{dias}).

FIG. 3A illustrates the generation of a baseline signal or waveform from a set of reference signals.

FIG. 3A shows a set of waveform representations 302 within a feature space 304. FIG. 3A further shows a first cluster 306, a second cluster 308, a third cluster 310, a fourth cluster 312, and a centroid 314.

The set of waveform representations 302 correspond to representations of individual waveforms or signals within the feature space 304. The waveforms are obtained from an individual over one or more periods of time at which they are in a steady state (e.g., prior to onset of hemodynamic instability or blood loss). In one embodiment, the waveforms are generated during an initial monitoring period. For example, in a clinical setting, this period could correspond to the first 10 minutes, 20 minutes, 30 minutes, or the like, after the device has started monitoring the patient.

In FIG. 3A, each integer “1”-“4” represents a single cardiac cycle waveform extracted from one or more coherent light-based signals. In one embodiment, the waveforms are dynamically time warped to have the same length such that each waveform representation shown in FIG. 3A corresponds to a high-dimensional representation of a waveform. Alternatively, the waveforms are mapped into the feature space 304 using a feature extraction, feature selection, or feature transformation algorithm (e.g., the feature selection approach described in FIG. 2, principal components analysis, latent feature encoding, etc.). The set of waveform representations 302 are then clustered within the feature space 304 to generate a set of N clusters. In the example shown in FIG. 3A, N=4 leading to the generation of the first cluster 306, the second cluster 308, the third cluster 310, and the fourth cluster 312.

The largest cluster may be considered representative of the steady state, or baseline state, of the individual. In FIG. 3A, the second cluster 308 corresponds to the largest cluster (i.e., the cluster having the greatest number of waveforms associated therewith). To determine a reference signal or waveform for the individual, the centroid (e.g., mean) of the largest cluster is calculated. In FIG. 3A, the centroid 314 corresponds to baseline or reference signal for the individual. The centroid 314 is then mapped back into the original space to generate the baseline signal. In embodiments where the set of waveform representations 302 correspond to high-dimensional representations of cardiac cycle signals, the centroid 314 is already in a form corresponding to the baseline signal.

FIG. 3B illustrates the generation of relative features according to embodiments of the present disclosure.

FIG. 3B shows a waveform under evaluation 316, a reference waveform 318, a feature extractor 320, a set of features 322, a set of reference features 324, a comparator 326, a set of relative features 328, and a prediction model 330.

The waveform under evaluation 316 corresponds to the waveform or signal currently being used to determine a hemodynamic state for an individual (e.g., a waveform extracted from the signal 118 shown in FIG. 1). The reference waveform 318 corresponds to a baseline or reference waveform for the individual. In one embodiment, the reference waveform 318 is generated using a clustering approach as described in relation to FIG. 3A above. In an alternative embodiment, the reference waveform 318 corresponds to a universal, or global, baseline waveform which is a synthetic waveform representing an idealized pulse shape.

The feature extractor 320 corresponds to the feature extractor 120 described above in relation to FIG. 1 which generates the set of features 322 from the waveform under evaluation 316 and the set of reference features 324 from the reference waveform 318. Example features extracted from these waveforms are shown in FIG. 2 as described above. The comparator 326 determines the set of relative features 328 from the set of features 322 and the set of reference features 324. For example, the set of relative features 328 may correspond to the percentage change of the set of features 322 from the set of reference features 324 (i.e., the increase or decrease in each feature value relative to the baseline encoded in the set of reference features 324). As an alternative example, the set of relative features 328 may correspond to the subtraction of the set of features 322 from the set of reference features 324.

The set of relative features 328 are then provided to the prediction model 330 (trained on relative feature values) to determine the hemodynamic state of the individual.

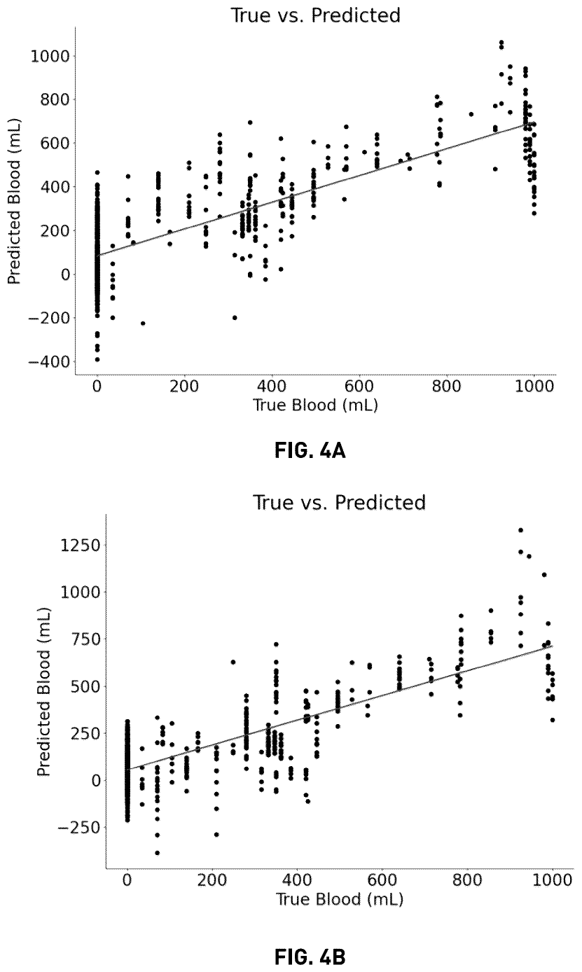

FIG. 4A illustrates true blood loss versus the blood loss predicted by the device 102 using absolute features (i.e., without relative normalization as described in relation to FIG. 3B). In this case, a Pearson's R of 0.80 and a mean error of 183 mL is observed.

FIG. 4B illustrates true blood loss versus blood loss predicted by the device 102 using relative features (i.e., using relative normalization as described in relation to FIG. 3B). In this case, a Pearson's R of 0.83 and a mean error of 163 mL is observed.

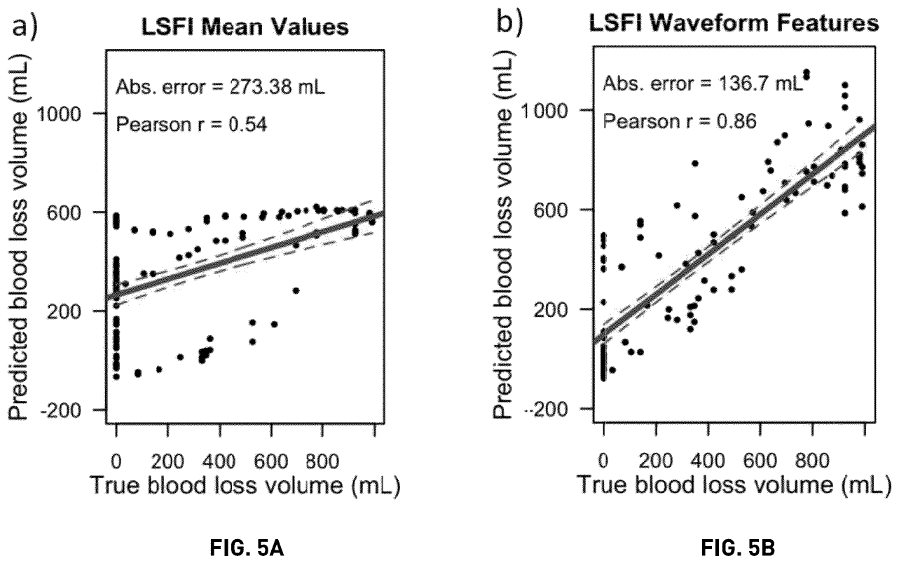

FIG. 5A illustrates the performance of estimating hemodynamic state of an individual using mean signal values obtained from a coherent light-based sensor. The hemodynamic state consists of a value indicative of blood loss volume and a linear regression model is used to predict the value from the mean signal values. This approach leads to an absolute error value of 273.38 mL and a Pearson's R of 0.54.

FIG. 5B illustrates the performance of estimating hemodynamic state of an individual using features extracted from signals generated by a coherent light-based sensor. The hemodynamic state consists of a value indicative of blood loss volume and a linear regression model is used to predict the value from the mean signal values. This approach leads to an absolute error value of 136.7 mL and a Pearson's R of 0.86. This is a significant improvement over the approach shown in FIG. 5A.

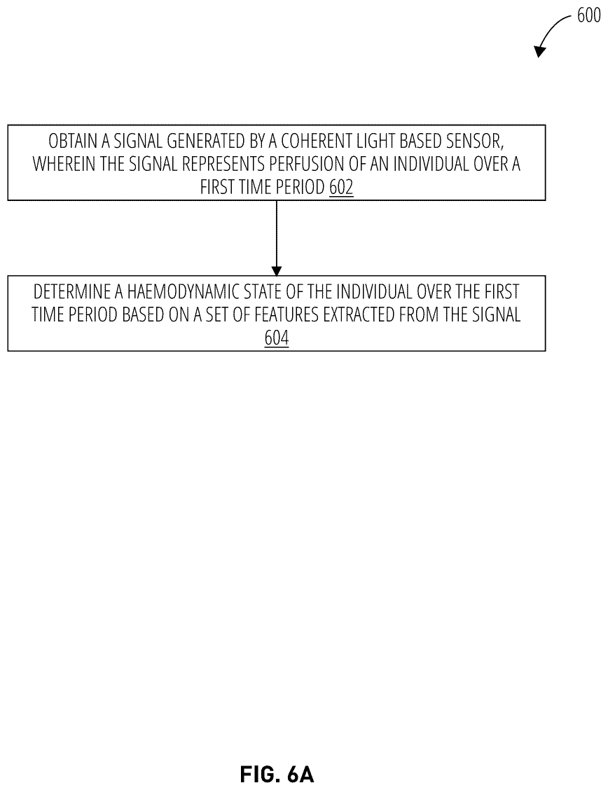

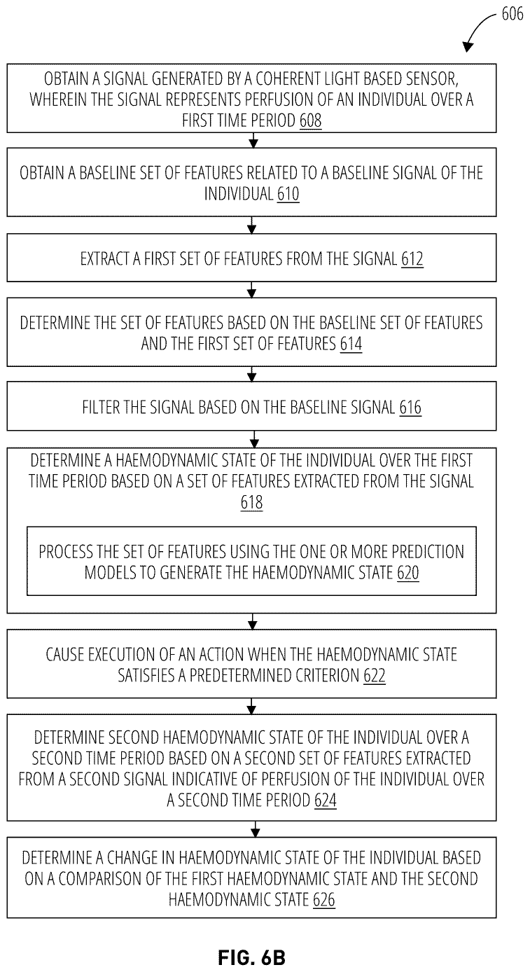

FIG. 6A shows a method 600 according to an aspect of the present disclosure.

At block 602, the method 600 may comprise obtaining a signal generated by a coherent light-based sensor. The signal represents perfusion of an individual over a first time period.

This step may be performed by a device such as the device 102 shown in FIG. 1. A coherent light-based sensor, which may be a speckleplethysmography (SPG) sensor, illuminates tissue of the individual with coherent optical radiation and generates an output signal indicative of blood flow based on light returned from the tissue. The signal may comprise a waveform including one or more cardiac pulses and may be pre-processed, for example, by being bandpass filtered prior to feature extraction.

At block 604, the method 600 may comprise determining a hemodynamic state of the individual over the first time period based on a set of features extracted from the signal. The step 604 may be performed by the device 102 or the second device 124 depending on the particular embodiment. That is, in some embodiments, the device 102 performs steps 602 and 604, while in some embodiments, the device 102 performs step 602 and the second device 124 performs step 604.

The set of features extracted from the signal may be morphological or pulsatile features that encode the shape, structure, and timing characteristics of the one or more cardiac pulses. As described in relation to FIG. 2, these features may include a systolic amplitude, a diastolic amplitude, a time delay, and various time and area values. This set of features may then be processed using one or more prediction models, such as statistical or machine learning models, to generate the hemodynamic state, which may comprise a value quantifying an amount of blood loss, a value indicative of cardiac function, or a value indicative of cardiovascular stability.

FIG. 6B shows a method 606 according to an aspect of the present disclosure.

At block 608, the method 606 may comprise obtaining a signal generated by a coherent light-based sensor, wherein the signal represents perfusion of an individual over a first time period. This step may be performed by the device 102. In one embodiment, block 608 corresponds to block 602 of the method 600 shown in FIG. 6A. The steps after step 608 may be performed by the device 102, the second device 124, or a combination of the two.

In some embodiments, the signal obtained at block 608 is pre-processed prior to further analysis and/or processing. As described in more detail above, this may involve, for example, bandpass filtering the to isolate the frequency range corresponding to cardiac activity and segmenting the signal to identify individual cardiac pulses, or waveforms, within the signal.

At block 610, the method 606 may comprise obtaining a baseline set of features related to a baseline signal of the individual.

The baseline signal encodes a characteristic or steady-state hemodynamic state of the individual. This baseline signal may be determined from a plurality of reference signals obtained from the individual, for example by using a clustering algorithm as shown in FIG. 3A.

At block 612, the method 606 may comprise extracting a first set of features from the signal.

A feature extractor, such as the feature extractor 120 shown in FIG. 1, may be used to extract the first set of features from the signal (or individual cardiac pulses segmented from the signal). In general, the set of features extracted from the signal describe the shape, structure, and timing characteristics of the signal.

At block 614, the method 606 may comprise determining the set of features based on the baseline set of features and the first set of features.

As illustrated in FIG. 3B, a comparator may be used to determine a set of relative features by comparing the baseline set of features to the first set of features (extracted from the current signal). This determination may involve calculating the difference or the percentage change between the corresponding features in the two sets.

At block 616, the method 606 may comprise filtering the signal based on the baseline signal.

The processing performed at block 616 may correspond to part of a quality control (QC) process, as described above. For example, the signal, or a part thereof such as a single cardiac pulse, may be compared to the baseline signal, which serves as a reference waveform. If the correlation between the signal and the baseline waveform is below a predetermined threshold, the features associated with that waveform are filtered out and excluded from subsequent processing.

At block 618, the method 606 may comprise determining a hemodynamic state of the individual over the first time period based on a set of features extracted from the signal. In one embodiment, block 618 corresponds to block 604 of the method 600 shown in FIG. 6A.

The set of features used at block 618 may be the first set of features (absolute features) extracted at block 612, or the set of relative features determined at block 614. The hemodynamic state may comprise various values indicative of the individual's cardiovascular condition, such as a value quantifying an amount of blood loss or a value indicative of cardiac function.

At block 620, the method 606 may comprise, as part of block 618, processing the set of features using the one or more prediction models to generate the hemodynamic state.

Prediction models (such as the one or more models 122 shown in FIG. 1) may be used to predict values indicative of the hemodynamic state of individual. These models may include linear regression models, random forest models, or deep learning models, and they may be trained on absolute feature values or relative feature values to generate the hemodynamic state output.

At block 622, the method 606 may comprise causing execution of an action when the hemodynamic state satisfies a predetermined criterion.

The action may be triggered when the hemodynamic state of the individual is indicative of a predetermined state such as a potential hemorrhage. The action may comprise generating an alert, such as a visual or audible alert, on the device (e.g., the device 102) or a device communicatively coupled to the device (e.g., the remote device 124). Alternatively, the action may involve updating one or more parameters of the coherent light-based sensor, such as increasing its sampling rate to improve signal resolution during a period of suspected instability.

At block 624, the method 606 may comprise determining a second hemodynamic state of the individual over a second time period based on a second set of features extracted from a second signal indicative of perfusion of the individual over a second time period.

At block 626, the method 606 may comprise determining a change in hemodynamic state of the individual based on a comparison of the first hemodynamic state and the second hemodynamic state.

This comparison allows for the tracking of the individual's hemodynamic trajectory over time. Determining this change in state can inform clinical tasks by indicating the extent to which the individual is compensating or decompensating, thereby providing an indication of potential risk. This rate of change may also be used as part of the criterion for causing execution of an action at block 622.

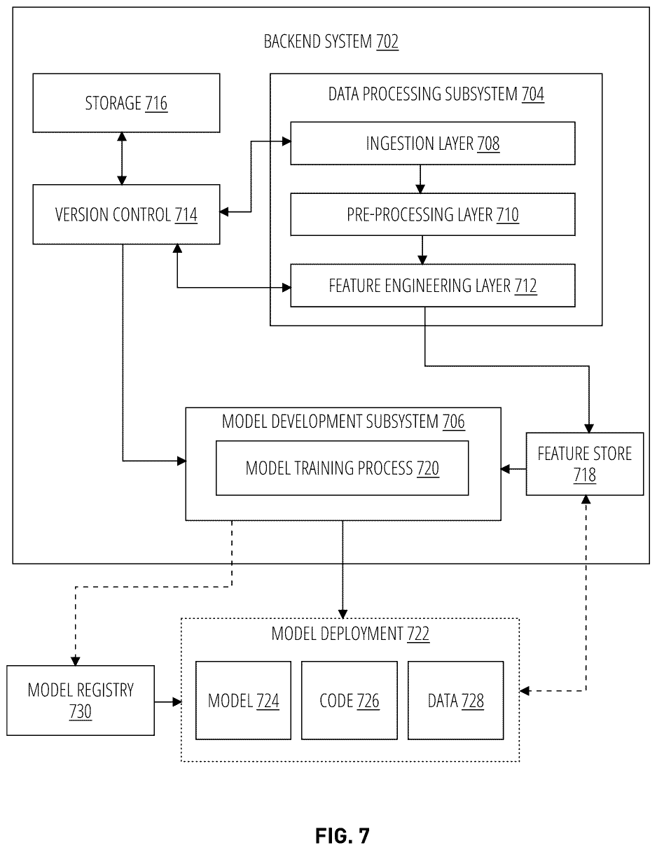

FIG. 7 illustrates a machine learning infrastructure that may be used in connection with the device 102, for example to perform the training and machine learning steps discussed above.

A backend system 702 coordinates data processing and model development. The backend system 702 comprises a data processing subsystem 704 and a model development subsystem 706. The data processing subsystem 704 comprises an ingestion layer 708, a pre-processing layer 710, and a feature engineering layer 712. A version control module 714 is coupled to a storage 716 which stores data sets. A feature store 718 is coupled to the data processing subsystem 704 and the model development subsystem 706. The model development subsystem 706 comprises a model training process 720. The ingestion layer 708 receives data via the version control module 714 and passes the data to the pre-processing layer 710. The pre-processing layer 710 passes processed data to the feature engineering layer 712. The data may then be stored in the storage 716 via the version control module 714. Engineered features may also be stored in the feature store 718. The model training process 720 trains a machine learning model on training data obtained from the storage 716 via the version control 714 or from the feature store 718. During a model deployment stage 722, a trained model 724 is deployed alongside code 726 and data 728. A model registry 730 performs version control and lineage tracking of the model 724 and data 728.

The ingestion layer 708 comprises processes and/or components for acquiring raw data (records) from files, object stores, databases, and message streams and materializes them into internal datasets. The pre-processing layer 710 comprises processes and/or components for performing various pre-processing tasks such as parsing, type casting, normalization, missing-value handling, deduplication, and data validation. The feature engineering layer 712 comprises processes and/or components for transforming processed inputs into features through joins, aggregations, window functions, and encoding functions (e.g., one-hot encoding, embeddings, etc.).

The version control module 714 tracks dataset manifests and links them to commits and tags. The version control module 714 can store large datasets and model artifacts in external object storage while keeping lightweight metadata under source control. The storage 716 can be a file system or object store that contains dataset files, intermediate artifacts, and saved models, with URIs recorded in a metadata store for lineage queries.

The feature store 718 provides structured storage and retrieval of engineered features for training and inference and can expose a common access layer to both the data processing subsystem 704 and the model development subsystem 706. The feature store 718 maintains an offline store for historical feature values used to build training datasets. The model development subsystem 706 orchestrates training, evaluation, and registration activities and runs the model training process 720 as a pipeline step. The model development subsystem 706 can distribute the model training process 720 by launching coordinated worker processes across multiple GPUs and servers, sharding batches for data-parallel execution and can also partition model layers for tensor or pipeline parallelism.

The model training process 720 loads training data either as datasets from the storage 716 referenced through the version control module 714 or as assembled training tables from the feature store 718. The model training process 720 is configured to train any suitable supervised machine learning algorithm. Example algorithms include linear regression, logistic regression, support vector machines with linear or kernelized decision functions, decision trees, ensemble methods such as random forests and gradient boosted trees, k-nearest neighbors, and neural networks including feedforward networks, convolutional networks, recurrent networks, and transformer encoders. The model training process 720 may employ any standard training approaches as known in the art. For example, a standard approach for training a machine learning model comprises obtaining relevant training data (e.g., as described above) and performing cross-validation to train the prediction model on the training data. For classification models, the cross-validation strategy can be stratified. Model hyperparameters can be selected using any suitable approach such as grid search or randomized search. Model performance can be estimated using any suitable performance measure and is dependent on the type of model being trained (e.g., mean square error for regression, binary cross entropy for classification, ranking loss for ranking, etc.).

At the model deployment stage 722, the trained model 724 may be packaged with code 726 and/or any required data 728 into a deployment unit and an inference interface may be subsequently exposed. The model 724 is a serialized artifact created by the model training process 720, stored in the storage 716 with an addressable uniform resource identifier (URI), and referenced by the model registry 730. The code 726 can comprise training code, feature computation code, and/or serving code. The data 728 comprises lookup tables and/or model assets that may be needed at inference. The model registry 730 may be used to store registered models, model versions, and metadata. The model registry 730 can also store documentation and lineage fields, including references to data 728 and feature groups used in training, to support governance and comparison across versions.

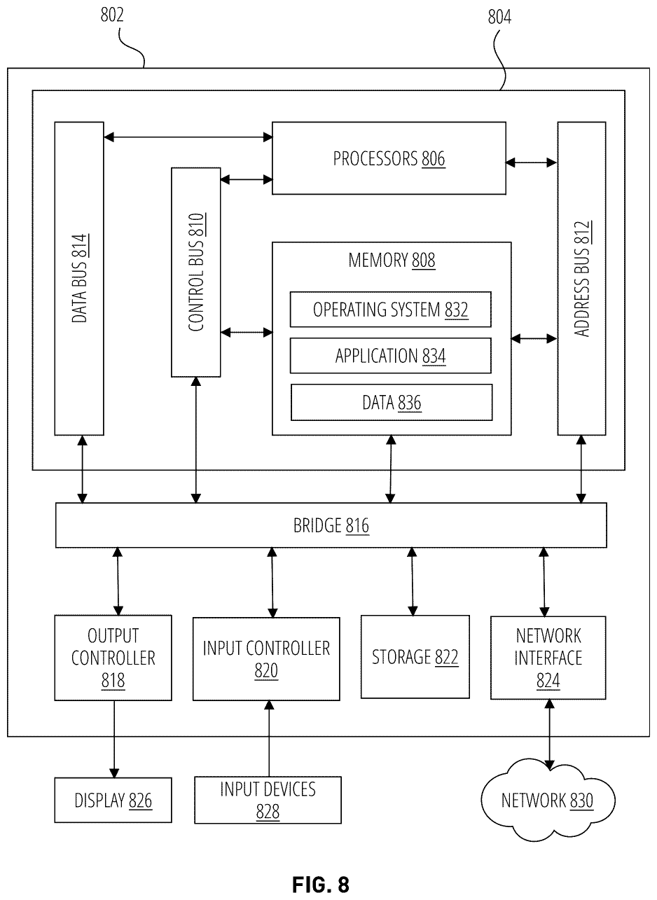

FIG. 8 shows a computing device 802 according to example implementations of the present disclosure. The computing device 802 may be configured to perform any of the operations of the present disclosure, such as those shown in relation to FIG. 1 to FIG. 7. The device 102, the second device 124, and the machine learning infrastructure may each include or use an implementation of the computing device 802 or a portion of the computing device 802. For example, the device 802 may include the computing device 802 and the processing circuitry 112, the memory 116, and the communication circuitry 114 of the device 102 may be implemented by computing device 802.

The computing device 802 comprises a central processing architecture 804, a bridge 816, an output controller 818, an input controller 820, a storage 822, and a network interface 824. The central processing architecture 804 includes one or more processors 806 coupled to the memory 808 via a control bus 810 and an address bus 814. The processors 806 are also coupled to a data bus 812. The control bus 810 transmits control signals that coordinate the operation of memory 808 and processors 806, including read/write commands and synchronization signals. The address bus 814 carries address information from processors 806 to memory 808, enabling the processors to specify memory locations for data access. The data bus 812 facilitates bidirectional transfer of data between processors 806 and other components, including peripheral subsystems.

The memory 808 is configured to store executable instructions and runtime data. It may include volatile memory such as dynamic random-access memory (DRAM) and static random-access memory (SRAM), as well as non-volatile memory elements. In use, the memory 808 may store the operating system 832, the application 834, and/or the data 836. The operating system 832 provides a runtime environment and resource management functions for the computing device 802. The operating system 832 may include kernel-level services for process scheduling, memory management, device I/O, and inter-process communication. Example operating systems include Microsoft Windows 10 or 11, Unix, Linux, and TempleOS. The application 834 executes within the context of the operating system 832 and may include user-level software modules configured to perform specific computational tasks. The data 836 may include structured or unstructured information accessed or generated by the application 834, and may be transferred from/to the storage 822.

The control bus 810, the data bus 812, the address bus 814, and the memory 808 are coupled to the bridge 816 which serves as a communication interface between the central processing architecture 804 and peripheral subsystems. The bridge 816 may perform protocol translation, data buffering, and arbitration functions to manage data flow across heterogeneous components.

The output controller 818 is configured to manage data transmission from the computing device 802 to the display 826. The output controller 818 may include frame buffers, timing generators, and digital-to-analog conversion circuitry and may be configured to format pixel data and synchronizes display refresh cycles. The display 826 may be a raster-based output device such as a liquid crystal display (LCD), organic light-emitting diode (OLED) panel, or other graphical interface. The input controller 820 receives signals from the input devices 828, which may include keyboards, pointing devices, touch-sensitive surfaces, or other human interface peripherals. The input controller 820 is configured to interpret electrical signals from the input devices 828 and convert them into digital data for processing by the processors 806 and/or other peripheral components/subsystems.

The storage 822 provides non-volatile data retention and may include magnetic disk drives, solid-state drives, or other persistent memory technologies. The storage 822 is coupled to the bridge 816 and supports read and write operations initiated by the processors 806 or other subsystems. It may store the operating system 832, the application 834, and the data 836 when not actively loaded into the memory 808.

The network interface 824 enables the computing device 802 to communicate with external systems via the network 830. The network interface 824 may support wired protocols such as Ethernet or serial communication, and/or wireless protocols such as Wi-Fi, Bluetooth, or cellular standards. The network 830 may include local area networks (LANs), wide area networks (WANs), or the Internet.

The computer, processor, and/or processing circuitry-implemented methods and processes described herein may include additional, fewer, or alternate actions. The present systems and methods may be implemented using one or more local or remote processors, transceivers, and/or sensors, and/or through implementation of computer-executable instructions stored on non-transitory computer-readable media or medium. Unless described herein to the contrary, the various steps of the several processes may be performed in a different order, or simultaneously in some instances.

Additionally, the computer devices and systems discussed herein may include additional, fewer, or alternative elements and respective functionalities, including those discussed elsewhere herein, which themselves may include or be implemented according to computer-executable instructions stored on non-transitory computer-readable media or medium.

The methods and systems may be implemented using computer programming or engineering techniques including computer software, firmware, hardware, or any combination or subset.

The above illustrative examples of various aspects and implementations provide an overview for understanding aspects and implementation of the disclosed method. The figures provided herein depict exemplary aspects of the present system and methods and are not intended to limit the scope of the disclosure.

Unless otherwise stated, all technical terms used herein have the same meaning as commonly understand by a person skilled in the art. Singular forms “a”, “an” and “the” include plural references unless the context of the disclosure clearly dictates otherwise. The term “or” is intended to encompass “and/or” unless clearly stated otherwise.

The terminology “coupled” used herein encompasses both a direct connection between two components/devices/systems, and an indirect electrical connection where the two components/devices/system are connected to each other via one or more intermediate components/devices/systems.

All publications, patents, and patent applications mentioned in this specification are herein incorporated by reference to the same extent as if each individual publication, patent, or patent application was specifically and individually indicated to be incorporated by reference.

What is claimed is:

1. A system comprising:
a coherent light-based sensor operable to generate a signal representing perfusion of an individual over a first time period; and
processing circuitry configured to determine a hemodynamic state of the individual over the first time period based on a set of features extracted from the signal.

2. The system of claim 1 wherein the hemodynamic state comprises a value indicative of one or more of: vascular and cardiac dynamics of the individual; an amount of blood loss by the individual; a blood volume of the individual; a degree of adequacy of tissue perfusion; a cardiac function of the individual over the first time period; a cardiovascular stability of the individual over the first time period; and onset of one or more of: preeclampsia, hemorrhage, postpartum hemorrhage.

3. The system of claim 1 wherein the processing circuitry is further configured to:
cause execution of an action when the hemodynamic state satisfies a criterion.

4. The system of claim 3 wherein the criterion is satisfied when the hemodynamic state is indicative of one or more of: onset of a potential hemorrhage; and impending onset of a potential hemorrhage.

5. The system of claim 3 wherein the action is an alert selected from one or more of: a visual alert; an audible alert; and a haptic alert.

6. The system of claim 5 wherein the system comprises a device including the coherent light-based sensor and the processing circuitry and the alert is executed on the device.

7. The system of claim 5 wherein the system comprises a first device including the coherent light-based sensor and a second device communicatively coupled to the first device, and the alert is executed on the second device.

8. The system of claim 3 wherein execution of the action updates one or more parameters of the coherent light-based sensor.

9. The system of claim 3 wherein the criterion is based on one or more of: a baseline physiology of the individual; a rate of change in hemodynamic state of the individual; and a compensation trajectory of the individual.

10. The system of claim 1 wherein the set of features comprises: one or more morphological features determined from the signal; frequency-based features determined from the signal; statistical features determined from the signal; and noise-based features determined from the signal.

11. The system of claim 1 wherein the processing circuitry is further configured to determine the set of features based on the signal.

12. The system of claim 11 wherein the set of features is determined relative to a baseline signal for the individual, wherein the baseline signal encodes a characteristic hemodynamic state of the individual.

13. The system of claim 12 wherein the baseline signal is determined from a plurality of reference signals obtained from the individual or is a synthetic signal.

14. The system of claim 13 wherein the baseline signal is determined from the plurality of reference signals obtained from the individual using a clustering algorithm.

15. The system of claim 1 wherein the processing circuitry is further configured to segment the signal to extract one or more cardiac pulses.

16. The system of claim 15 wherein the set of features is based on at least one of the one or more cardiac pulses.

17. The system of claim 1 wherein the processing circuitry is further configured to:
process the set of features using one or more prediction models to determine the hemodynamic state, and the one or more prediction models comprise one or more of:
a linear regression model; a random forest model; and a deep learning model.

18. The system of claim 1 wherein the hemodynamic state is determined based on the set of features and one or more demographic features and/or electronic health record features related to the individual.

19. The system of claim 1 wherein the coherent light-based sensor is one of: a speckleplethysmography (SPG) sensor; a speckle contrast optical spectroscopy (SCOS) sensor; a dynamic light scattering (DLS) sensor; and a speckle visible spectroscopy sensor.

20. The system of claim 1 further comprising a wearable device including the coherent light-based sensor.

21. The system of claim 1 wherein the hemodynamic state is a first hemodynamic state and the processing circuitry is further configured to:
determine a second hemodynamic state of the individual over a second time period based on a second set of features extracted from a second signal indicative of perfusion of the individual over a second time period; and
determine a change in hemodynamic state of the individual based on a comparison of the first hemodynamic state and the second hemodynamic state.

22. The system of claim 1, further comprising a device including the coherent light-based sensor and the processing circuitry.

23. The system of claim 1, further comprising a first device including the coherent light-based sensor, and a second device the processing circuitry remote from the first device.

24. A method comprising:
obtaining, by processing circuitry, a signal generated by a coherent light-based sensor, wherein the signal represents perfusion of an individual over a first time period; and
determining, by processing circuitry, a hemodynamic state of the individual over the first time period based on a set of features extracted from the signal.

25. A non-transitory computer readable medium including instructions which, when executed by processing circuitry causes the processing circuitry to perform the method of claim 24.
